(19) United States
(12) Patent Application Publication
(10) Pub. No.: US 2016/0030182 A1
(43) Pub. Date: Feb. 4, 2016

(54) PROSTHETIC COMPONENT WITH CROSSLINKED POLYMER WEAR ZONE AND EDGE PROTECTION

(71) Applicant: Derek James Wallace MCMINN, (US)

(72) Inventor: Derek James Wallace McMinn, West Midlands (GB)

(21) Appl. No.: 14/647,597

(22) PCT Filed: Apr. 17, 2014
(86) PCT No.: PCT/GB2014/051211

§ 371 Date: May 27, 2015

(30) Foreign Application Priority Data
Apr. 25, 2013 (GB) 1307451.3

(51) Int. Cl. A61F 2/30; B29B 11/14; B29C 43/18; A61F 2/38; A61F 2/34

(52) U.S. Cl. CPC A61F 2/3094; A61F 2/389; A61F 2/34; A61F 2/3877; B29C 43/183; B29B 11/14; B29K 2023/06; B29K 2105/24

(57) ABSTRACT

A method of manufacturing a prosthetic component comprises providing crosslinked polymer in a mould and moulding non-crosslinked polymer to a free edge of the crosslinked polymer to form a hybrid component. A portion of the crosslinked polymer and a portion of the non-crosslinked polymer is then removed so as to form a prosthetic component having a full thickness crosslinked wear zone and at least one non-crosslinked edge.

### FIELD OF THE INVENTION

The present invention relates to prosthetic component with a crosslinked polymer wear zone and edge protection. In particular embodiments, the prosthetic component may constitute the whole or a part of a prosthesis, for example, an acetabular cup prosthesis for use in hip resurfacing or a tibial component for use in a knee replacement procedure.

### BACKGROUND TO THE INVENTION

It is known to use a metal acetabular cup with a separate crosslinked polymer inner liner in a hip replacement or resurfacing procedure. It is also known to direct compression mould non-crosslinked polymer power to a metal cup shell to create a monoblock component and then to crosslink the polymer portion by radiation.

Due to its superior wear properties when compared to conventional polyethylene, it has further been proposed to use a thin layer of crosslinked polyethylene to form an articular bearing surface of a prosthetic component and to leave a bulk layer behind the crosslinked polyethylene, which is not crosslinked, and which is moulded to a metal cup shell so as to retain good mechanical properties.

Where a thin crosslinked polyethylene articular layer is provided in an acetabular cup prosthesis (either as a thin homogenous crosslinked layer or alternatively a thin crosslinked surface layer on top of a conventional polyethylene layer, as described above), it has the disadvantage that loads applied to the cup edge can fracture the crosslinked polyethylene layer resulting in increased debris and a high likelihood of pain and further complications.

The same principle applies to knee implants. However, in this case, the weak crosslinked polyethylene layer has the additional disadvantage that failure can occur at a locking mechanism provided between the crosslinked polyethylene layer and a metal tibial base plate and fracture of associated stabilising pegs can also occur.

Shoulder and other implants (e.g. dual mobility hip bearings) including crosslinked polyethylene layers have similar disadvantages of vulnerability to edge loading fracture and/or failure at locking mechanisms.

It is therefore an aim of the present invention to provide a prosthetic component with a crosslinked polymer wear zone and edge protection, which helps to ameliorate some of all of the afore-mentioned problems.

### SUMMARY OF THE INVENTION

According to a first aspect of the present invention there is provided a method of manufacturing a prosthetic component comprising:
providing crosslinked polymer in a mould;
moulding non-crosslinked polymer to a free edge of the crosslinked polymer to form a hybrid component; and
removing a portion of the crosslinked polymer and a portion of the non-crosslinked polymer so as to form a prosthetic component having a full thickness crosslinked wear zone and at least one non-crosslinked edge.

Embodiments of this aspect of the invention therefore provide an effective method for producing thin prosthetic components (e.g. for othopaedic implants) that have a full thickness crosslinked wear zone and a non-crosslinked edge for increased resistance to damage through edge loading. It will be understood that the term ‘full thickness’ is intended to denote that a transverse cross-section through the wear zone will reveal only crosslinked polymer (i.e. there is no non-crosslinked polymer backing or support material).

The method may comprise direct compression moulding of the prosthetic component. More preferably, the method may comprise the manufacture of a plurality of prosthetic components in a bulk moulding process. In such an embodiment, a plurality of crosslinked polymer constituents may be provided in the mould; non-crosslinked polymer may be moulded to a free edge of each of the crosslinked polymer constituents to form a plurality of hybrid components; and a portion of each of the crosslinked polymer constituents and a portion of the non-crosslinked polymer on each hybrid component may be removed so as to form a plurality of prosthetic components, each having a full thickness crosslinked wear zone and at least one non-crosslinked edge. This has the advantage of producing many more prosthetic components in a quicker and more cost-effective manner than would otherwise be achievable with, for example, direct compression moulding (DCM) techniques. Advantageously, no bespoke tooling is required and a variety of different sizes or shapes of components can be produced at once. For example, it is possible for the present method to be employed to produce approximately 2,200 components from a single tool, within a 24 hour period.

The method may further comprise moulding the crosslinked and/or non-crosslinked polymer to a metal base. This step may be performed at substantially the same time as the non-crosslinked polymer is moulded to the free edge of the crosslinked polymer. Alternatively, the hybrid component or the formed prosthetic component may be subsequently moulded to the metal base or subsequently attached by melting an exterior surface of the crosslinked and/or non-crosslinked polymer so that it attaches to a roughened, undercut or porous surface of the metal base when solidified (e.g. by heating the metal base using ultrasound, inductive heating or standard thermal heating of the metal base in an oven).

In other embodiments, the method may comprise attaching the crosslinked and/or non-crosslinked polymer to a metal base via a fixation mechanism. For example, a simple mechanical or frictional locking mechanism may be employed in which the elasticity of the polymer is exploited by forcing the prosthetic component into a prepared recess in the metal base at the time of surgery.

The metal base may be porous.

The metal base may be constituted by, for example, an acetabular cup shell or a base plate for a tibial or patellar component.

The crosslinked polymer may comprise an antioxidant (e.g. Vitamin E).

The crosslinked polymer may be provided in the form of a consolidated preform (e.g. one which has been hot compression moulded). Alternatively, the crosslinked polymer may be provided in the mould in the form of irradiated and compressed (e.g. cold compressed) polymer powder.

Although, the edge of the crosslinked polymer may be substantially planar, it is preferred that the edge includes a bevel to increase the surface area of the edge to maximise the area of contact with the non-crosslinked polymer and/or to provide a larger space to accommodate non-crosslinked polymer at the edge of the component for increased strength.

The compressed or preformed crosslinked polymer may have a smooth, digitated or roughened edge to help in fusing the crosslinked polymer to the non-crosslinked polymer on moulding.

The preform may be moulded prior to being radiation crosslinked. Alternatively, the preform may be formed from irradiated polymer powder.

In either case, the antioxidant may be infused into the preform after radiation crosslinking.

In certain embodiments, the antioxidant may be blended with polymer powder prior to compression of the polymer powder and/or prior to moulding of the preform.

The non-crosslinked polymer may comprise an antioxidant (e.g. Vitamin E).

The non-crosslinked polymer may be provided in powder form. Alternatively, the non-crosslinked polymer may be provided in the form of a consolidated preform. The non-crosslinked preform may be shaped to mate with the crosslinked polymer provided in the mould.

The method may further comprise one or more of the following steps: machining, cleaning, packing and sterilising the prosthetic component.

If antioxidant is provided in the non-crosslinked polymer, the sterilising step may comprise terminal radiation sterilisation. This can be helpful to regain full crosslinking of the wear zone since a reduction in crosslinking may occur as a result of the moulding process. It will be understood that, during terminal radiation sterilisation some crosslinking of the non-crosslinked edge may occur if the concentration of antioxidant is not sufficient to inhibit radiation induced crosslinking. It is therefore desirable that a sufficient concentration of antioxidant is present in the non-crosslinked polymer so as to eliminate (or at least minimise) any crosslinked resulting from terminal radiation sterilisation. Conversely, the level of antioxidant present in the crosslinked polymer may be determined so as to allow further crosslinking upon terminal radiation sterilisation whilst eliminating (or at least minimising) the free radicals resulting from such further crosslinking.

It is preferable that terminal radiation sterilisation is not employed in embodiments where the non-crosslinked polymer contains no antioxidant since, in this case, there is no mechanism to minimise crosslinking of the non-crosslinked polymer and, as a result, the desirable strength properties of the non-crosslinked polymer edge may be lost. Furthermore, if terminal radiation sterilisation is employed when the edge polymer does not contain antioxidant, free radicals will result which makes the edge prone to oxidation in use.

The step of moulding the non-crosslinked polymer to a free edge of the crosslinked polymer may comprise compression moulding. For example, hot compression moulding may be employed to melt the free edge of the crosslinked polymer and the non-crosslinked polymeric material so that they fuse together on cooling.

Where the prosthetic component is configured as an acetabular cup bearing component, a metal cup may be positioned in a part-spherical (e.g. hemi-spherical) mould and a crosslinked polymer preform may be located in the metal cup. The prefrom may comprise a part-spherical external surface terminating a distance from the edge of the cup and having an internal trunk extending vertically from the pole of the cup. Where the acetabular cup is configured for resurfacing (as opposed to THR), the metal cup may comprise an inferior cutout and the the trunk may merge into an inferior edge of the preform and a groove may be provided between the trunk and a superior edge of the preform to minimise flow of the preform under gravity when in a molten state during moulding. Non-crosslinked polymer powder may then be placed around the preform to completely fill the exposed portions around the edges of the metal cup. Non-crosslinked powder may also be provided above the preform (and in the groove in the preform) so as to provide a substantially horizontal surface layer on which a top plate (i.e. platen) of the mould can be provided to compress and mould the elements together.

After moulding (e.g. fusing), the metal cup and moulded polymer layers can be removed from the mould and a cutting tool employed to remove the portions of the crosslinked polymer and non-crosslinked polymer required so as to form a prosthetic component having a full thickness crosslinked wear zone and at least one non-crosslinked edge mounted on the metal cup. It will be understood that the metal cup will provide a reference for the removal of the required portions of polymer.

Where the prosthetic component is configured as a dual mobility hip component, a crosslinked polymer prefrom may be located in a mould having a short cylindrical recess extending vertically downwardly from the centre of a part-spherical (e.g. hemi-spherical) recess. The preform may comprise a complementary cylindrical locating spigot depending from a solid part-spherical body. An upper horizontal surface of the body may be substantially flat or may include a roughened or digitated edge. Non-crosslinked polymer powder may then be placed on top of the preform to completely fill any gaps in the roughened or digitated edge and to build up a layer of non-crosslinked powder above the preform so as to provide a substantially horizontal surface layer on which a top plate of the mould can be provided to compress and mould the polymers together.

After moulding, the hybrid component may be removed from the mould and a cutting tool employed to remove the portions of the crosslinked polymer and non-crosslinked polymer required so as to form a prosthetic component having a full thickness crosslinked wear zone and at least one non-crosslinked edge mounted on the metal cup. For example, a part-spherical recess may be formed in the upper surface of the body and the spigot removed to provide a hollow part-spherical component having an inner and and outer crosslinked wear zone and a free edge of non-crosslinked polymer.

Where the prosthetic component is configured as a tibial component, a metal base having a planar upper surface and two hollow legs depending therefrom may be positioned in mould having complementary recesses and a crosslinked polymer preform comprising a solid complementary body with depending legs may be located on the metal base. The prefrom is sized so as to leave an edge of the metal base exposed. Non-crosslinked polymer powder may then be placed around and on top of the preform to completely fill the exposed portions around the edge of the metal base and to build up a layer of non-crosslinked powder above the preform so as to provide a substantially horizontal surface layer on which a top plate of the mould can be provided to compress and mould the polymers and the base together.

After moulding, the hybrid component attached to the base may be removed from the mould and a cutting tool employed to remove the portions of the crosslinked polymer and non-crosslinked polymer required so as to form a prosthetic component having a full thickness crosslinked wear zone and at least one non-crosslinked edge around the metal base.

The crosslinked polymer may be formed by chemical or radiation crosslinking of a polymeric starting material. The polymeric starting material may be in the form of powder, flakes, resin or a consolidation.

The preforms may be consolidated by compression moulding, direct compression moulding, or ram extrusion. The preforms may be irradiated to induce crosslinking before, during or after consolidation.

In particular embodiments, the preforms may be formed by machining a compression moulded bar stock of polymer particles, wherein the bar stock has been irradiated to induce crosslinking before, during or after moulding.

In the case of the crosslinked polymer, the antioxidant may be introduced to (e.g. blended with or doped into) the polymer before, during or after crosslinking. It should be noted that the presence of an antioxidant will help to reduce oxidation. Accordingly, the method of the present invention may be carried out in an oxygen-containing atmosphere (e.g. air) as the risk of oxidation (by combination of oxygen with free radicals during the moulding process) will be minimised due to the presence of the antioxidant. This also means that large moulding apparatus (e.g. presses) can be employed to produce many components at once since tight control of the surrounding environmental conditions is not required (i.e. it is not necessary to perform the method in a vacuum or inert environment). Furthermore, the presence of antioxidant in the crosslinked polymer material means that relatively long moulding cycles (up to 24 hours) can be used without increasing the risk of oxidation of the preforms.

In an embodiment, 0.1% by weight of vitamin E was blended with polymer powder and the mixture was then irradiated with a dose of 150 kGy to provide a sufficiently crosslinked polymer powder which was then subsequently consolidated and formed into the preforms.

In another embodiment, polymer powder may be consolidated (either directly into the preforms or into a bar stock from which the preforms are derived) and then doped by diffusion of an antioxidant, before, during or after the consolidation is irradiated to induce crosslinking.

Where the antioxidant is blended with polymer particles, the antioxidant should substantially coat the surfaces of all of the polymer particles present. The polymer particles may be provided in the form of a resin (e.g. comprising powder, flakes and/or small pellets) or a hydrogel (e.g. comprising a polymer capable of absorbing water). The polymer particles may comprise a plurality of molecules.

The crosslinked or non-crosslinked polymer may comprise the following but is not limited thereto: polyethylene, polypropylene, polyamide, polyimide, polyether ketone, or any polyolefin, including high-density-polyethylene, low-density-polyethylene, linear-low-density-polyethylene, ultra-high molecular weight polyethylene (UHMWPE), copolymers and mixtures thereof; hydrogels such as poly(vinyl alcohol), poly(ethylene glycol), poly(ethylene oxide), poly(acrylic acid), poly(methacrylic acid), poly(acrylamide), copolymers and mixtures thereof; copolymers and mixtures of a hydrogels with any polyolefin.

The antioxidant may be provided in the form of a liquid, powder, solution or suspension. For example, a powder (or liquid) antioxidant may be dissolved in a solvent such as alcohol to increase the volume of the antioxidant containing element and allow it to more easily coat the polymer particles. The solvent may be evaporated off after the blending. Alternatively, for example for insoluble antioxidants, the bulk of the antioxidant containing element can be increased by placing the antioxidant in a suspension of liquid (e.g. water).

The antioxidant may comprise the following but is not limited thereto: vitamin E; alpha-tocopherol, delta-tocopherol; propyl, octyl, or dedocyl gallates; lactic, citric, ascorbic, tartaric acids; organic acids and their salts; orthophosphates; tocopherol acetate; Irganox 1010 or other hindered amines.

In certain embodiments of the present invention, the antioxidant (e.g. vitamin E) may constitute up to 3% by weight or volume of the polymer. In particular embodiments, the antioxidant (e.g. vitamin E) may constitute 0.1%, 0.5%, 1%, 2%, or 3% by weight or volume of the polymer.

The prosthetic component may be suitable for use in any joint, for example in acetabular cups, tibial and patella components of knees, spine disc replacements, shoulders, jaws, ankles, toes, elbows, wrists, fingers or thumbs.

According to a second aspect of the present invention there is provided a prosthetic component comprising a full thickness crosslinked wear zone and at least one non-crosslinked edge.

The prosthetic component may be manufactured by the method described above in relation to the first aspect of the invention.

### BRIEF DESCRIPTION OF THE DRAWINGS

Embodiments of the invention will now be described, by way of example only, with reference to the accompanying figures, in which:

FIG. 1 shows an exploded perspective view of the initial steps in a method for manufacturing a prosthetic component in the form of a resurfacing acetabular cup, in accordance with a first embodiment of the invention;

FIG. 2 shows an exploded view of the setup in FIG. 1 after a metal cup has been placed in a mould;

FIG. 3 shows the mould of FIG. 2 after a crosslinked preform has be located in the metal cup in the mould;

FIG. 4 shows a cross-sectional view through the setup of FIG. 3 after non-crosslinked powder has been placed over the mould and a mould plate positioned on top;

FIG. 5 shows a cross-sectional view of the acetabular cup resulting from the setup in FIG. 4 after portions of the crosslinked and non-crosslinked polymer have been removed;

FIG. 6 shows a perspective view of the acetabular cup of FIG. 5;

FIG. 7 shows a view similar to that of FIG. 4 but wherein the mould and crosslinked preform are configured for the production of a dual mobility hip component;

FIG. 8 shows a cross-sectional view of the dual mobility hip component resulting from the setup in FIG. 7 after portions of the crosslinked and non-crosslinked polymer have been removed;

FIG. 9 shows a view similar to that of FIG. 4 but wherein the mould, metal base and crosslinked preform are configured for the production of a tibial component;

FIG. 10 shows a front-to-back cross-sectional view of the tibial component after it has been removed from the setup in FIG. 9 and partially machined into shape; and

FIG. 11 shows a cross-sectional view of the tibial component of FIG. 10 after further machining to remove portions of the crosslinked and non-crosslinked polymer.

### DETAILED DESCRIPTION OF CERTAIN EMBODIMENTS

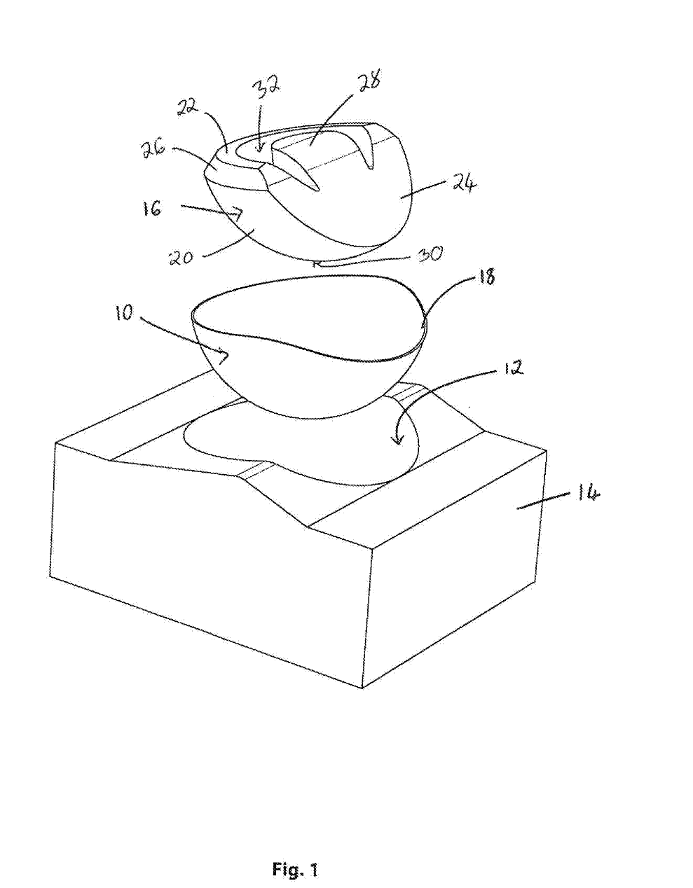

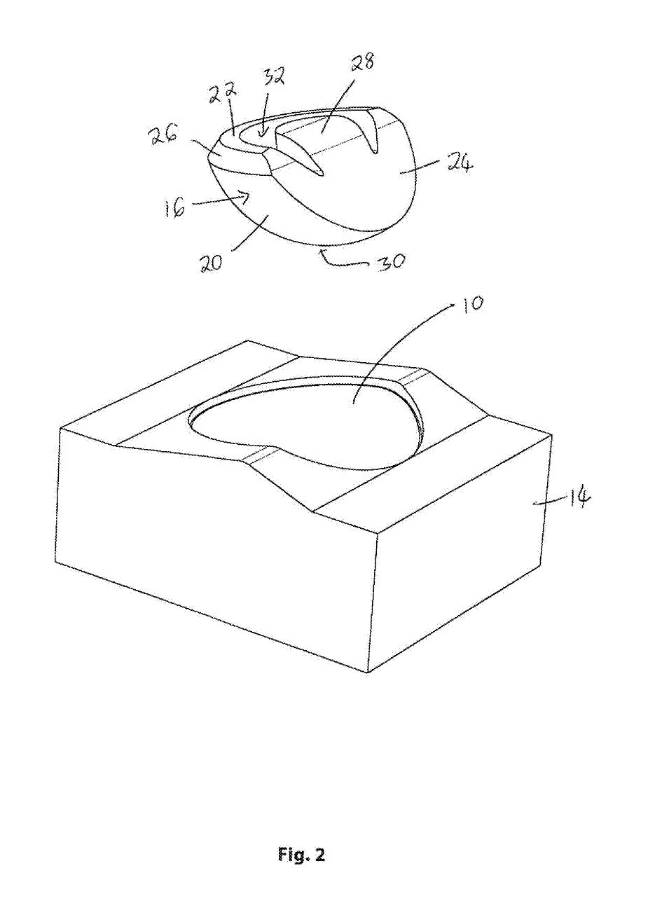

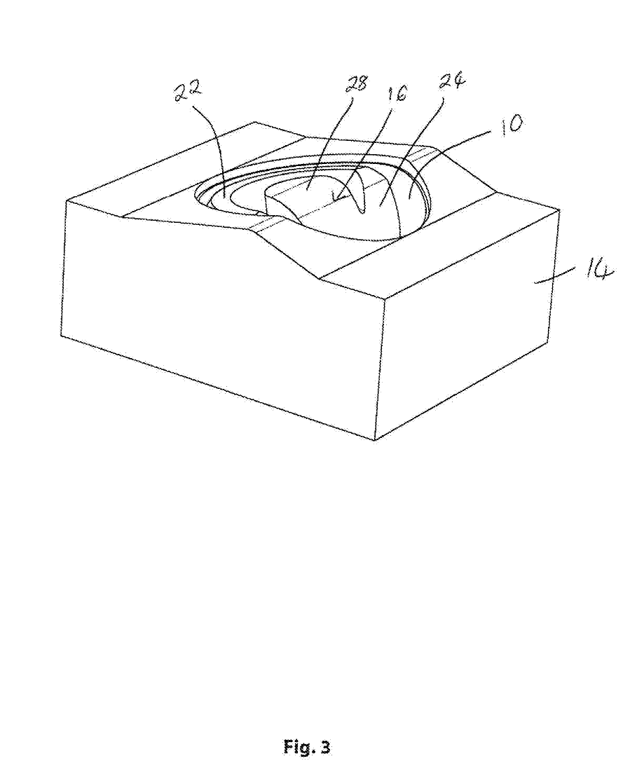

FIGS. 1 to 3 illustrate the initial steps in a method for manufacturing a prosthetic component in the form of an acetabular cup for use in hip resurfacing, in accordance with a first embodiment of the invention. In this embodiment, the method comprises positioning a metal cup 10 within a part-sperical recess 12 in a mould base 14 and locating a crosslinked polymer preform 16 in the metal cup 10.

The metal cup 10 is formed of porous titanium (although in other embodiments titanium alloy, tantalum or cobalt chromium alloys may be employed), is substantially hemi-spherical and includes an anterior-inferior cut-out 18.

The prefrom 16 is formed from polyethylene powder which has been blended with vitamin E, consolidated and radiation crosslinked. The prefrom 16 comprises a part-spherical external surface 20 terminating at an upper superior edge 22 and a lower inferior edge 24. The superior edge 22 includes a bevel 26 to increase the surface area for attachment to non-crosslinked polymer, as will be described below. The preform 16 also has an internal trunk 28 extending vertically from the pole 30 of the cup. The trunk 16 merges into the inferior edge 24 and a groove 32 is provided between the trunk 28 and the superior edge 22 to aid correct location and orientation of the preform 16 in the metal cup 10.

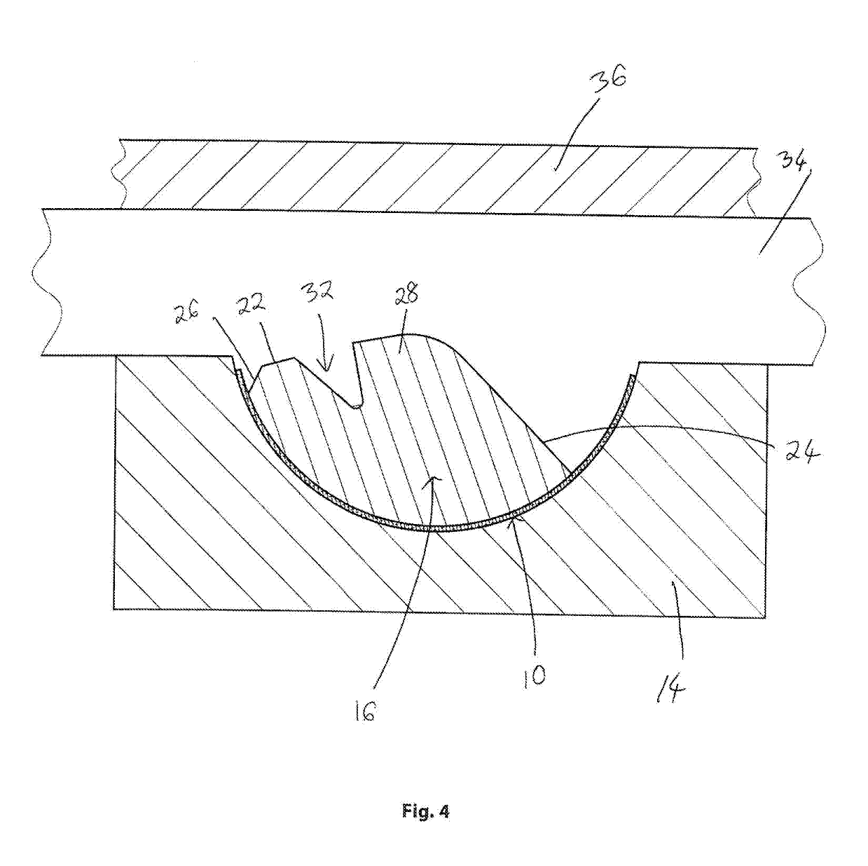

As shown in FIG. 4, non-crosslinked polyethylene powder 34 (which has also been blended with vitamin E) is placed around the preform 16 to completely fill the exposed portions around the edges of the metal cup 10. In addition, the non-crosslinked powder 34 is provided above the preform 16 (and in the groove 32 in the preform 16) so as to provide a substantially horizontal surface layer on which a top plate 36 of the mould is provided to compress and mould the elements together.

It will be understood that the mould base 14 and top plate 36 may be made of metal (e.g. stainless steel or aluminium) or a polymer (e.g. silicone rubber) which has a melting point above the moulding temperature of the crosslinked and non-crosslinked polymers 16, 34. For example, a moulding temperature of around 190° C. may be used to fuse the non-crosslinked polymer 34 to the crosslinked preform 16 and also to fuse the polymers to the porous metal cup 10.

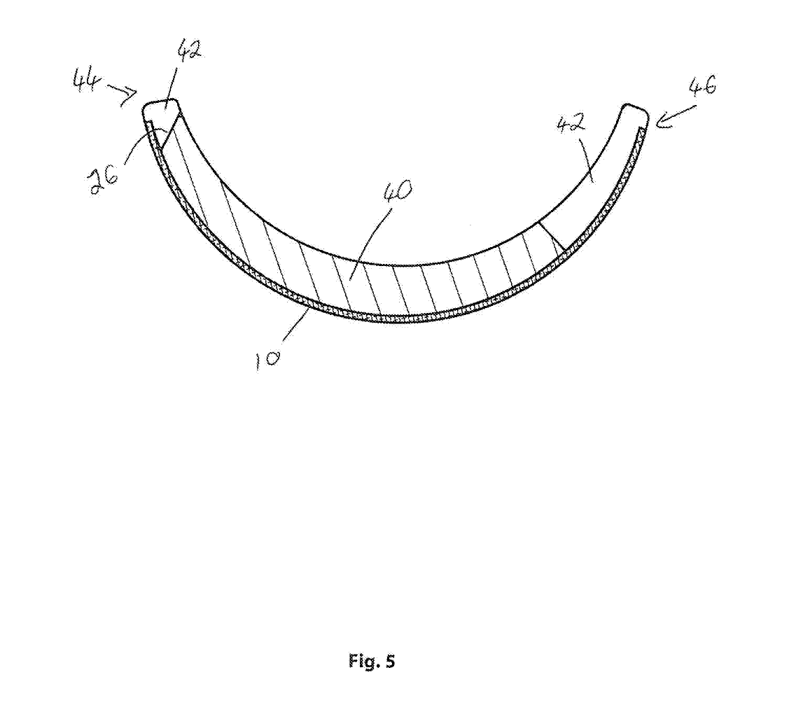

After moulding, the metal cup 10 with attached polymers is removed from the mould base 14 and a cutting tool is employed to remove the portions of the crosslinked polymer 16 and non-crosslinked polymer 34 required so as to form a prosthetic component as illustrated in FIG. 5. In particular, FIG. 5 shows a superior-inferior cross-section of a machined final acetabular cup component having a full thickness crosslinked wear zone 40 and a non-crosslinked edge 42 mounted on the metal cup 10. In this view, it can be seen that the non-crosslinked edge 42 in the superior region 44 of the cup 10 is smaller than the non-crosslinked edge 42 in the inferior region 46 of the cup 10. It will also be noted that the bevel 26 provided on the prefrom 16 provides a larger area than would otherwise be provided for accommodating the non-crosslinked polymer 42 in the superior region 44 of the cup 10.

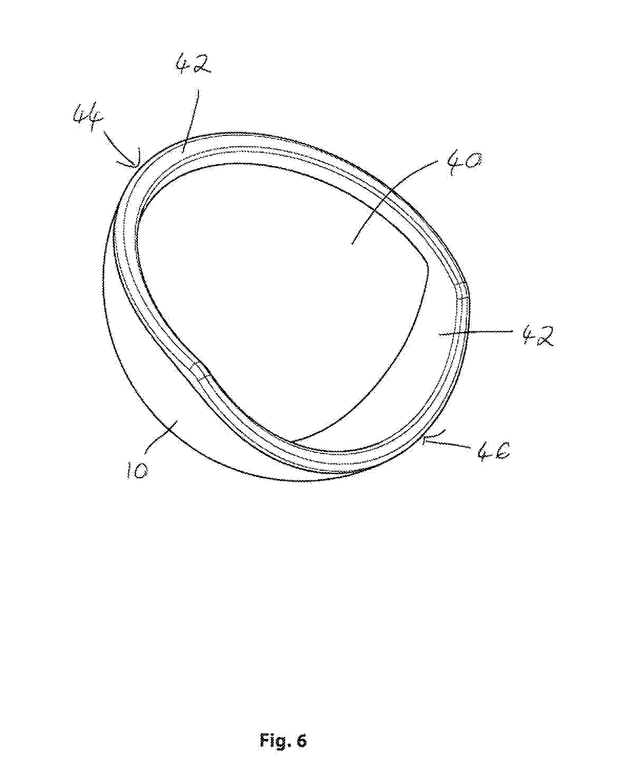

FIG. 6 shows a perspective view of the component illustrating the continuous nature of the non-crosslinked edge 42. In this embodiment, the prosthetic component is subjected to terminal radiation sterilisation to enhance the crosslinking in the wear zone after moulding.

It will be understood that the method described above may also be employed to produce an acetabular cup for a total hip replacement (THR). In which case, the cup will typically have a 180° articular surface, without an inferior cut-out, and the polymer liner (including a full thickness crosslinked wear zone and non-crosslinked edge) will be attached to the metal shell via a mechanical peripheral locking mechanism. Thus, a moulding setup similar to that illustrated in FIG. 4 may be employed but wherein the preform is symmetrical as there is no need to accommodate an inferior cut-out.

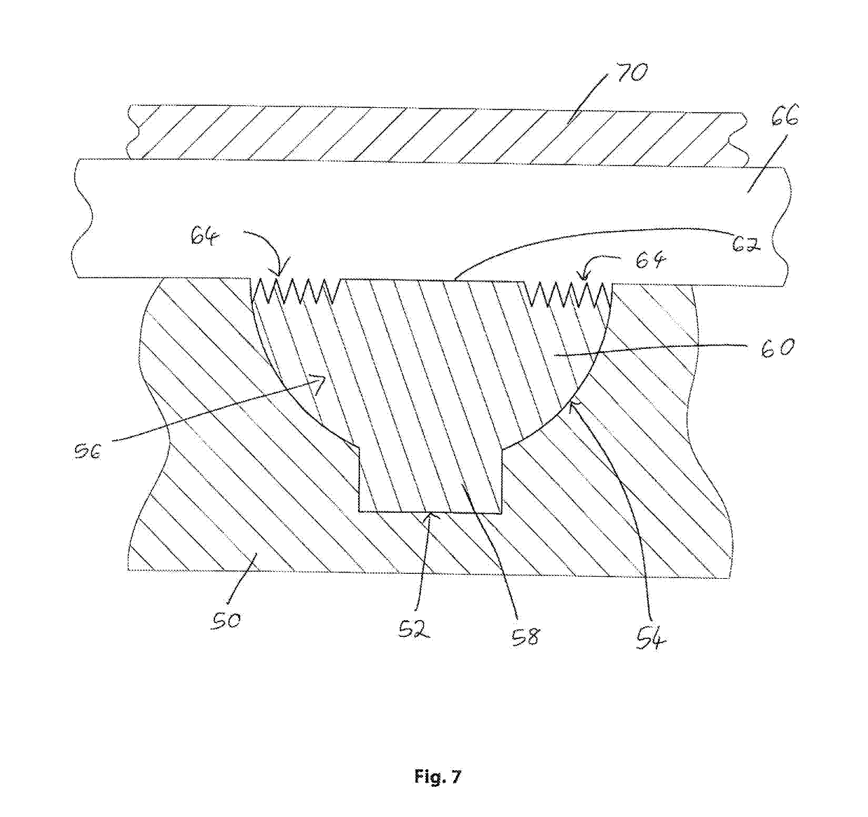

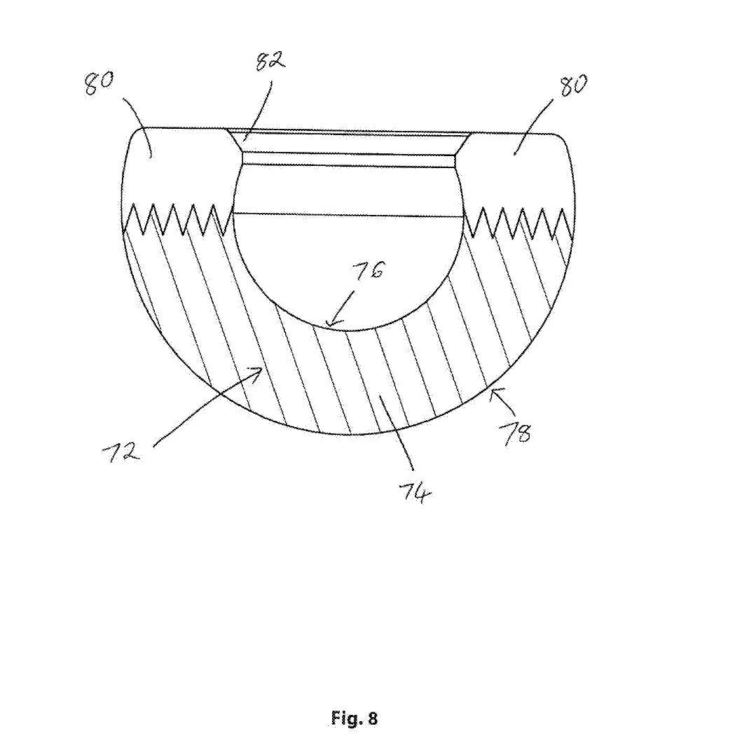

FIGS. 7 and 8 illustrate a method for manufacturing a prosthetic component in the form of a dual mobility hip component, in accordance with a second embodiment of the invention. In this embodiment, the method is similar to that described above but no metal components are required. In this instance, a mould base 50 is provided which includes a short cylindrical recess 52 extending vertically downwardly from the centre of a hemi-spherical recess 54. A crosslinked preform 56 having a complementary cylindrical locating spigot 58 depending from a solid part-spherical body 60 is then located in the mould base 50. The spigot 58 is provided to prevent the preform 56 from tilting when non-crosslinked polymer powder is poured on top prior to moulding. As above, the prefrom 56 is formed from polyethylene powder which has been blended with vitamin E, consolidated and radiation crosslinked. An upper horizontal surface 62 of the body 60 is substantially flat in the centre but has a digitated edge 64.

Non-crosslinked polymer powder 66 (preferably blended with vitamin E) is placed on top of the preform 56 to completely fill the gaps in the digitated edge 64 and to build up a layer of non-crosslinked powder above the preform 56 so as to provide a substantially horizontal surface layer on which a top plate 70 of the mould is provided to compress and mould the polymers together.

After moulding, the hybrid polymer component is removed from the mould base 50 and a cutting tool employed to remove the portions of the crosslinked polymer 56 and non-crosslinked polymer 66 required so as to form a prosthetic component as shown in FIG. 8. Thus, a part-spherical recess is formed in the upper surface 62 of the body 60 and the spigot 58 removed to provide a hollow part-spherical component 72 having a full thickness crosslinked wear zone 74 with an inner and an outer crosslinked articular surface 76, 78 and a free edge of non-crosslinked polymer 80. An inner surface 82 of the edge 80 may be beveled, as shown in FIG. 8, in order to increase the length of the edge which impinges on a femoral neck during use so as to provide line contact instead of the more dangerous point contact.

As above, the component 72 may be subjected to terminal radiation sterilisation. In which case, even if when the non-crosslinked polymer contains vitamin E, there may be some crosslinking of the edge 80. However, it will be understood that this will be minimised by the provision of vitamin E so that the edge retains good strength properties. This is particularly important in dual mobility hip components where impingement of the edge 80 on a femoral neck is common during use. In addition, as the inner surface of the component is often greater that 180°, the component must be snap-fitted onto a prosthetic femoral head component so that it is effectively retained thereon. In both of these cases translational forces may be applied to the edge 80 and therefore the use of a roughened or inter-digitated interface between the edge 80 and the wear zone 74 is advantageous to maintain the integrity of the component.

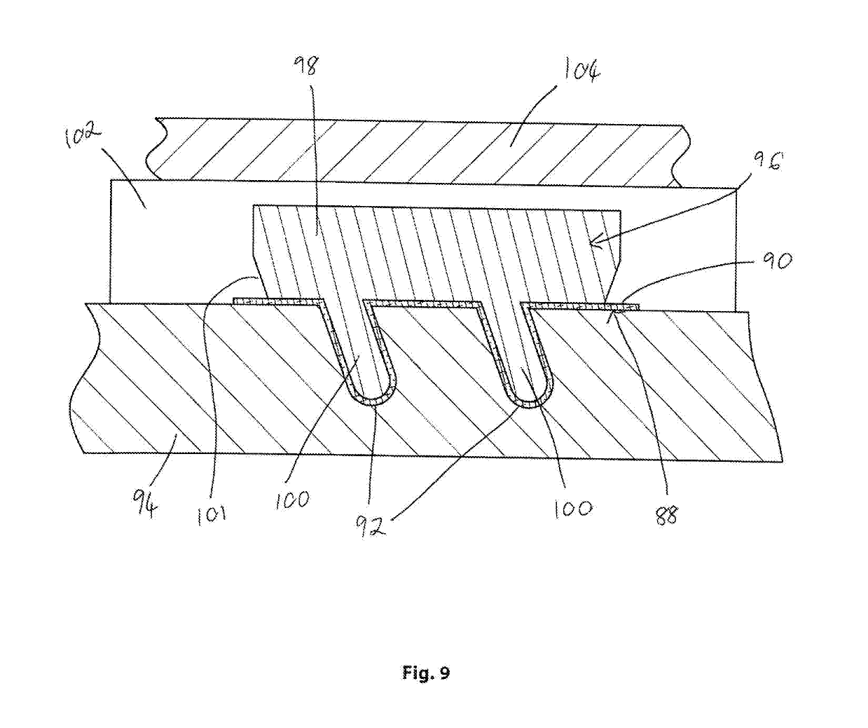

FIGS. 9 to 11 illustrate a method for manufacturing a prosthetic component in the form of a uni-compartmental tibial component for a knee (UKR), in accordance with a third embodiment of the invention. In this embodiment, the method comprises positioning a porous metal base plate 88 having a planar upper surface 90 and two hollow legs 92 depending (at an angle front-to-back) therefrom in mould base 94 having complementary recesses. A crosslinked polymer preform 96 comprising a solid complementary body 98 with depending legs 100 is then located on the metal base plate 88. The prefrom 96 (including vitamin E) is sized so as to leave an edge of the metal base plate 88 exposed. In addition, a lowermost corner of the body 98 is provided with a bevel 101 to increase the space available for accommodating non-crosslinked polymer 102 at the edge.

The non-crosslinked polymer powder 102 (preferably also including vitamin E) is then placed around and on top of the preform 96 to completely fill the exposed portions around the edge of the metal base plate 88 and to build up a layer of non-crosslinked powder 102 above the preform 96 so as to provide a substantially horizontal surface layer on which a top plate 104 of the mould is provided to compress and mould the polymers and the base plate 88 together.

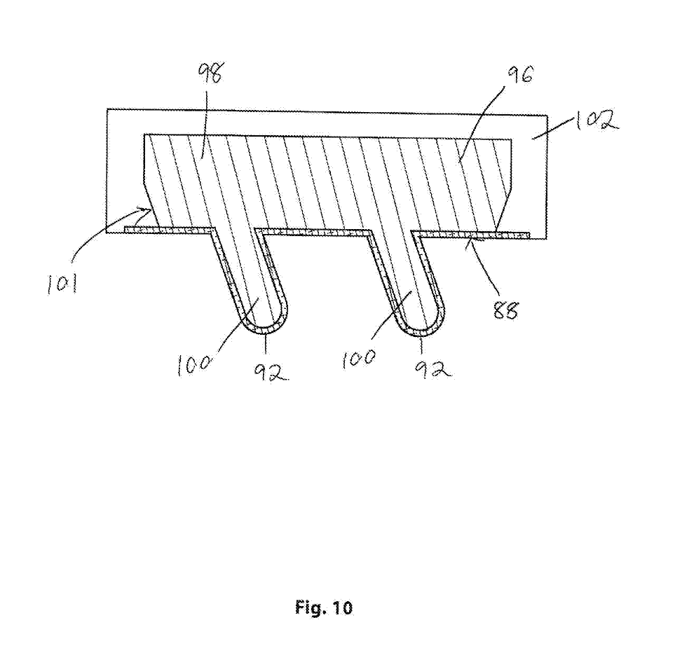

FIG. 10 shows a front-to-back cross-sectional view of the tibial component after it has been removed from the mould in FIG. 9 and partially machined into shape but prior to removal of any crosslinked polymer 96.

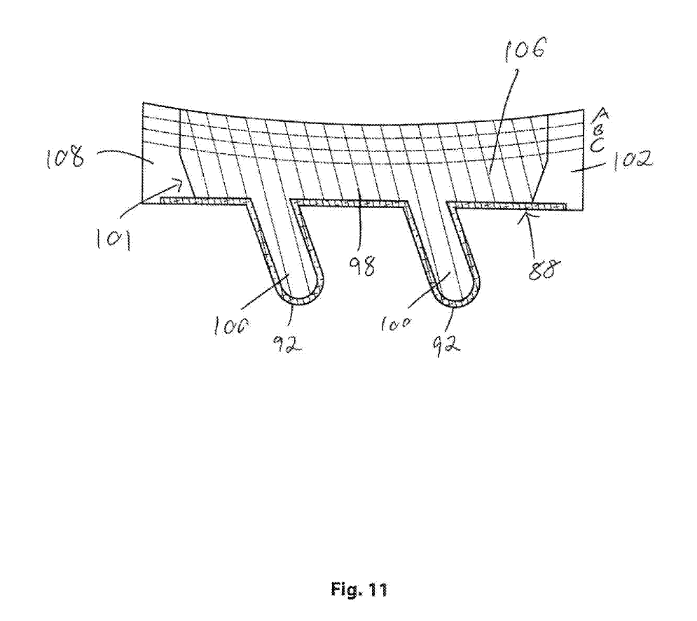

FIG. 11 shows the tibial component after further machining to remove portions of the crosslinked and non-crosslinked polymer 96, 102 so as to form a prosthetic component having a full thickness crosslinked wear zone 106 and a non-crosslinked edge 108. The dotted lines A, B, C represent optional thicknesses, which the component may be machined to as desired.

Alternatively, a tibial component for UKR may be made as two-piece components and the hybrid polymer component may be subsequently fixed (e.g. during surgery) to a metal base plate. In which case, the method described above would be carried out without the metal base plate and then the hybrid component would be attached by mechanical means to a metal base plate having holes for receipt of the legs 100. Alternatively, a commonly used peripheral mechanical locking mechanism may be employed to affix the hybrid component to the metal base plate.

In other embodiments of the invention, the above methods may be adapted to produce other components such as total knee replacement components (e.g. medial and lateral tibial components), patellar components, glenoid components for shoulder replacements or other components including those for use in ankle, toe, wrist, elbow or finger joints.

Although the methods shown in relation to each of the above embodiments concern the manufacture of only one prosthetic component at a time, it will be understood that, in practice, the moulds may be extended and may include multiple recesses for the simultaneous manufacture of multiple prosthetic components in an array.

It will be appreciated by persons skilled in the art that various modifications may be made to the above embodiments without departing from the scope of the present invention. For example, features described in relation to one embodiment may be mixed and matched with features described in relation to one or more other embodiments.

1. A method of manufacturing a prosthetic component comprising:
providing crosslinked polymer in a mould;
moulding non-crosslinked polymer to a free edge of the crosslinked polymer to form a hybrid component; and
removing a portion of the crosslinked polymer and a portion of the non-crosslinked polymer so as to form a prosthetic component having a full thickness crosslinked wear zone and at least one non-crosslinked edge.

2. The method according to claim 1 wherein:
a plurality of crosslinked polymer constituents are provided in the mould;
non-crosslinked polymer is moulded to a free edge of each of the crosslinked polymer constituents to form a plurality of hybrid components; and
a portion of each of the crosslinked polymer constituents and a portion of the non-crosslinked polymer on each hybrid component is removed so as to form a plurality of prosthetic components, each having a full thickness crosslinked wear zone and at least one non-crosslinked edge.

3. The method according to claim 1 further comprising moulding the crosslinked and/or non-crosslinked polymer to a metal base.

4. The method according to claim 3 wherein the step of moulding the crosslinked and/or non-crosslinked polymer to a metal base is performed at substantially the same time as the non-crosslinked polymer is moulded to the free edge of the crosslinked polymer.

5.-6. (canceled)

7. The method according to claim 3 wherein the metal base is constituted by an acetabular cup shell or a base plate for a tibial or patellar component.

8. The method according to claim 1 wherein the crosslinked polymer comprises an antioxidant.

9. The method according to claim 1 wherein the crosslinked polymer is provided in the form of a consolidated preform.

10. The method according to claim 1 wherein the crosslinked polymer is provided in the mould in the form of irradiated and compressed polymer powder.

11. The method according to claim 1 wherein the edge of the crosslinked polymer includes a bevel.

12. The method according to claim 1 wherein the compressed or preformed crosslinked polymer has a digitated or roughened edge to help in fusing the crosslinked polymer to the non-crosslinked polymer on moulding.

13. The method according to claim 9 wherein the preform is moulded prior to being radiation crosslinked.

14. The method according to claim 9 wherein the preform is formed from irradiated polymer powder.

15. The method according to claim 13 wherein antioxidant is infused into the preform after radiation crosslinking.

16. The method according to claim 13 wherein antioxidant is blended with polymer powder prior to compression of the polymer powder and/or prior to moulding of the preform.

17. The method according to claim 1 wherein the non-crosslinked polymer comprises an antioxidant.

18. The method according to claim 1 wherein the non-crosslinked polymer is provided in powder form.

19. The method according to claim 1 wherein the non-crosslinked polymer is provided in the form of a consolidated preform.

20.-22. (canceled)

23. The method according to claim 1 wherein the prosthetic component is configured as an acetabular cup bearing component.

24. The method according to claim 23 wherein the non-crosslinked inferior edge of the prosthetic component extends further towards the centre of the acetabular cup than the non-crosslinked superior edge.

25.-26. (canceled)

27. The method according to claim 1 wherein the prosthetic component is configured for use in a hip, knee, spine, shoulder, jaw, ankle, toe, elbow, wrist, finger or thumb.

28. A prosthetic component comprising a full thickness crosslinked wear zone moulded to at least one non-crosslinked edge.

29.-31. (canceled)
